(19) United States
(12) Patent Application Publication
(10) Pub. No.: US 2024/0000747 A1
(43) Pub. Date: Jan. 4, 2024

(54) SIRTUIN 6 ACTIVATOR

(71) Applicants: DAICEL CORPORATION, Osaka-shi, Osaka (JP); KYUSHU UNIVERSITY, NATIONAL UNIVERSITY CORPORATION, Fukuoka-shi, Fukuoka (JP)

(72) Inventors: Hiroaki YAMAMOTO, Tokyo (JP); Yoshinori KATAKURA, Fukuoka-shi (JP)

(73) Assignees: DAICEL CORPORATION, Osaka-shi, Osaka (JP); KYUSHU UNIVERSITY, NATIONAL UNIVERSITY CORPORATION, Fukuoka-shi, Fukuoka (JP)

(21) Appl. No.: 18/038,821

(22) PCT Filed: Nov. 26, 2021
(86) PCT No.: PCT/JP2021/043488

§ 371 Date: May 25, 2023

(30) Foreign Application Priority Data
Nov. 26, 2020 (JP) 2020-195763

(51) Int. Cl. A61K 31/366

(52) U.S. Cl. CPC A61K 31/366

(57) ABSTRACT

An object of the present invention is to provide a novel agent capable of activating SIRT6. A sirtuin 6 activator containing a urolithin as an active ingredient.

### TECHNICAL FIELD

The present disclosure relates to a sirtuin 6 activator, and more specifically, the present disclosure relates to a sirtuin 6 activator for use in pharmaceuticals, quasi-drugs, cosmetics, or foods.

### BACKGROUND ART

In studies on the control of aging and lifespan, the involvement of Sir2, a sirtuin having NAD-dependent deacetylase activity, has attracted attention. SIRT1 to SIRT7 are known as mammalian homologs of yeast Sir2. Among these, SIRT6 is localized in cell nuclei (e.g., of human keratinocytes and skin fibroblasts), and SIRT6 protein translated from SIRT6 gene has been reported to be useful as an early index marker of aging (Non-Patent Document 1).

For the purpose of enhancing the action and effect of SIRT6, several SIRT6 activators have been reported. Specifically, carnosine and/or anserine (Patent Document 1) and a peptide composed of an amino acid sequence of GAGVSAE-NH 2 (Patent Document 2) have been reported as active ingredients of SIRT6 activators.

Meanwhile, urolithins, such as urolithin A and urolithin C, are known as metabolites of ellagic acid derived from ellagitannins contained in pomegranates, raspberries, blackberries, cloudberries, strawberries, walnuts, and the like. In addition, actions and effects known for urolithin A include an antioxidant action (Non-Patent Document 2), an anti-inflammatory action (Non-Patent Document 3), and an anti-glycation action (Non-Patent Document 4).

Furthermore, SIRT1 is known to be involved in the regulation of enhancement of fat mobilization, inhibition of neuroaxonal degeneration, insulin secretion from cells, hepatic gluconeogenesis, and the like; and urolithin A, punicalin, puricalagin, cafestol, and the like are known to enhance the SIRT1 expression level (Patent Document 3).

### CITATION LIST

### Patent Documents

Patent Document 1: JP 2015-097508 A
Patent Document 2: WO 2016/094073
Patent Document 3: WO 2014/042261

### Non-Patent Document

Non-Patent Document 1: “Circulating SIRT6 Expression, Effect on Aging Quality (Sirt6)”, [online], retrieved on Jan. 30, 2020, U.S. National Institutes of Health, the Internet <URL:https://clinicaltrials.gov/ct2/show/record/NCT01567176>
Non-Patent Document 2: Biosci. Biotechnol. Biochem. 76, 395-399 (2012)
Non-Patent Document 3: J. Agric. Food Chem. 60, 8866-8876 (2012)
Non-Patent Document 4: Mol. Nutr. Food Res. 55, S35-S43 (2011)

### SUMMARY OF INVENTION

### Technical Problem

Among the SIRT6 activators reported so far, for example, carnosine produces (3-alanine, which can cause numbness, during digestion, anserine has a characteristic unpleasant odor (fishy odor) derived from a raw material, and certain peptides cannot be taken orally. In view of these, yet another new alternative is desired to be available for an active ingredient capable of activating SIRT6.

Thus, an object of the present disclosure is to provide a novel agent capable of activating SIRT6.

### Solution to Problem

The inventor has examined SIRT6 gene activity-enhancing effect of urolithin A, punicalin, puricalagin, and cafestol, which are known as components capable of enhancing the SIRT1 gene activity, and found that the urolithin has an enhancing action on the sirtuin 6 gene activity, while no or little enhancing effect on the SIRT6 gene activity was observed for punicalin, puricalagin, and cafestol. The present disclosure was completed through further examinations based on this finding.

That is, the present disclosure provides the invention of aspects listed below.
Item 1. A sirtuin 6 activator containing a urolithin as an active ingredient.
Item 2. The sirtuin 6 activator according to item 1, in which the urolithin is urolithin A.
Item 3. The sirtuin 6 activator according to item 1 or 2, for use as a therapeutic or prophylactic agent for hair loss.
Item 4. The sirtuin 6 activator according to item 1 or 2, for use as a hair follicle stem cell aging inhibitor.
Item 5. The sirtuin 6 activator according to item 1 or 2, for use as a therapeutic or prophylactic agent for gray hair.
Item 6. A therapeutic or prophylactic agent for hair loss, the agent containing a urolithin as an active ingredient.
Item 7. A therapeutic or prophylactic agent for gray hair, the agent containing a urolithin as an active ingredient.
Item 8. Use of a urolithin for producing a sirtuin 6 activator.
Item 9. A method for treating or preventing hair loss, the method including administering a therapeutically or prophylactically effective amount of a urolithin to a subject with a disease or condition in which hair loss occurs, or a subject at risk of hair loss due to a genetic factor or a physical factor.
Item 10. A method for treating or preventing gray hair, the method including administering a therapeutically or prophylactically effective amount of a urolithin to a subject in which gray hair occurs, or a subject with a subject at risk of hair graying due to a genetic factor or a physical factor.

### Advantageous Effects of Invention

According to the present disclosure, there is provided a novel agent capable of activating SIRT6.

### BRIEF DESCRIPTION OF DRAWINGS

FIG. 1 is a graph indicating effects on promoter activity of SIRT6 when urolithin A was added to cells into which a promoter of sirtuin 6 gene had been introduced.

### DESCRIPTION OF EMBODIMENTS

The present disclosure relates to a sirtuin 6 activator containing a urolithin as an active ingredient. Hereinafter, embodiments of the sirtuin 6 activator of the present disclosure will be described in detail.

### Active Ingredient

A sirtuin 6 activator according to the present disclosure contains a urolithin as an active ingredient. Urolithins are known components that are known to have actions, such as antioxidation, anti-inflammation, and anti-glycation.

The urolithin used in the present disclosure is not limited to a particular type. In one embodiment of the present disclosure, examples of the urolithin include substances represented by General Formula (1) below:

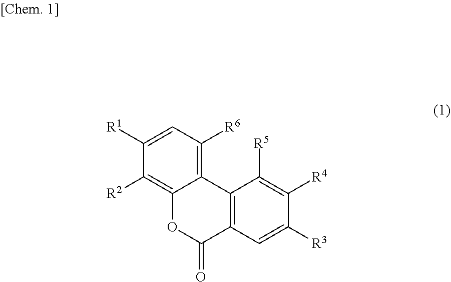

where in Formula (1), R¹ to R⁶ may each be the same or different and represent a hydrogen atom, a hydroxyl group, or a methoxy group.

Preferred examples of the urolithin include urolithin A, urolithin B, urolithin C, urolithin D, urolithin E, urolithin M3, urolithin M4, urolithin M5, urolithin M6, urolithin M7, and isourolithin A, in which R¹ to R⁶ in Formula (1) are groups respectively shown in Table 1 below.

TABLE 1

| | R¹ | R² | R³ | R⁴ | R⁵ | R⁶ |
|---|---|---|---|---|---|---|
| Urolithin A | —OH | —H | —OH | —H | —H | —H |
| Urolithin B | —OH | —H | —H | —H | —H | —H |
| Urolithin C | —OH | —H | —OH | —OH | —H | —H |
| Urolithin D | —OH | —OH | —OH | —OH | —H | —H |
| Urolithin E | —OH | —OH | —OH | —H | —OH | —H |
| Urolithin M3 | —OH | —H | —OH | —OMe | —H | —H |
| Urolithin M4 | —OH | —H | —OMe | —OH | —H | —H |
| Urolithin M5 | —OH | —OH | —OH | —OH | —OH | —H |
| Urolithin M6 | —OH | —H | —OH | —OH | —OH | —H |
| Urolithin M7 | —OH | —H | —OH | —H | —OH | —H |
| Isourolithin A | —OH | —H | —H | —OH | —H | —H |

One of these urolithins may be used individually, or two or more may be used in combination.

Among these urolithins, urolithin A is preferred from the viewpoint of obtaining an even better enhancing effect on the sirtuin 6 gene activity.

The method for synthesizing a urolithin is not limited to a particular method, and examples include chemical synthesis and microbiological synthesis.

Examples of the method for the chemical synthesis of a urolithin include a method in which 2-bromo-5-methoxybenzoic acid is used as a starting material and converted to 2-bromo-5-hydroxybenzoic acid by demethylation, which is further reacted with resorcinol to obtain urolithin A.

Examples of the method for the microbiological synthesis of a urolithin include a method in which a urolithin is produced by a urolithin-producing microorganism from ellagic acid by fermentation in a medium containing ellagic acid, and the produced urolithins are collected to obtain a urolithin. The urolithin-producing microorganism is not limited to a particular species or strain, but examples preferably include bacteria of the genus Bacteroides and bacteria of the genus Gordonibacter, more preferably include bacteria belonging to Bacteroides uniformis, bacteria belonging to Gordonibacter urolithinfaciens, and bacteria belonging to Gordonibacter pamelaeae, and even more preferably include Bacteroides uniformis HGB5B146 (NITE BP-02193) strain, Gordonibacter urolithinfaciens DSM27213 strain, and Gordonibacter pamelaeae DSM19378 strain.

In the sirtuin 6 activator of the present disclosure, the content of a urolithin is an amount that enables administration or ingestion of an effective amount for enhancing sirtuin 6 gene activity in vivo and is not limited to a particular amount. In the sirtuin 6 activator in one embodiment of the present disclosure, the content of a urolithin can be appropriately adjusted according to its application, dosage form, administration form, or the like. Specific examples include from 0.01 to 50 wt. % and preferably from 0.1 to 20 wt. %.

### Applications

The sirtuin 6 activator according to the present disclosure is for use in applications for activating sirtuin 6 (SIRT6). Activating SIRT6 means enhancing the activity of the SIRT6 gene and more specifically enhancing the transcription or expression of the SIRT6 gene without being limited to either in vivo or in vitro.

The presence or absence and degree of activation action of SIRT6 can be evaluated by constructing a system using the promoter region (SEQ ID NO: 1) of SIRT6 gene.

The sirtuin 6 activator in one embodiment of the present disclosure can be used for administering a urolithin to a subject for which activation of SIRT6 is desired for treating a disease or condition (including a pathological condition and a non-pathological condition, where the non-pathological condition includes, for example, conditions associated with physical activity and/or exercise in healthy subjects) associated with reduced or absent activity of SIRT6.

More specifically, the sirtuin 6 activator in one embodiment of the present disclosure can be used for applications based on the activation of sirtuin 6, including treatment or prevention of hair loss, inhibition of hair follicle stem cell aging, and/or treatment or prevention of gray hair. That is, in one embodiment of the present disclosure, there is also provided a therapeutic or prophylactic agent for hair loss, the agent containing a urolithin as an active ingredient, a hair follicle stem cell aging inhibitor containing a urolithin as an active ingredient, and a therapeutic or prophylactic agent for gray hair, the agent containing a urolithin as an active ingredient.

A specific subject to which the therapeutic or prophylactic agent for hair loss is applied in one embodiment of the present disclosure is any subject with a disease or a condition in which hair loss actually occurs, or any subject at risk of hair loss due to a genetic factor or a physical factor. Examples of the disease or condition in which hair loss occurs include alopecia other than androgenetic alopecia, and specific examples include female pattern alopecia and alopecia areata. In addition, examples of the risk of hair loss include possession of an alopecia areata risk genotype (specifically, T allele of SNP rs142986308 present in the CCHCR1 gene), chemotherapy treatment, hypervitaminosis A, malnutrition (e.g., iron deficiency and zinc deficiency, etc.), and stress (specifically, physical or mental stress associated with high fever, surgery, illness, weight loss, pregnancy, or the like).

A specific subject to which the hair follicle stem cell aging inhibitor and the therapeutic or prophylactic agent for gray hair in one embodiment of the present disclosure are applied is any subject in which aging of hair follicle stem cells or gray hair actually occurs, or any subject at risk of hair follicle stem cell aging or risk of hair graying due to a genetic factor or a physical factor. Examples of the risk of hair follicle stem cell aging or the risk of hair graying due to a genetic factor include possession of a hair graying risk genotype (specifically, T allele of SNP rs12203592 present in intron 4 of interferon regulatory factor 4 gene (IRF4)). In addition, examples of the risk of hair follicle stem cell aging or the risk of hair graying due to a physical factor include poor blood circulation in the scalp (e.g., a reddish scalp) and mental stress.

The sirtuin 6 activator in one embodiment of the present disclosure can be administered or ingested orally or parenterally (e.g., transdermally, transmucosally (such as nasally or enterally), or intravascularly (intraarterially or intravenously)) to a subject for which activation of SIRT6 is desired.

The dosage or intake of the sirtuin 6 activator of the present disclosure to a subject for which activation of SIRT6 is desired is appropriately set by those skilled in the art according to age, weight, sex, a disease or condition to which the sirtuin 6 activator is applied, and the like of the subject. For example, the dosage or intake of the sirtuin 6 activator in one embodiment of the present disclosure includes, for example, from 0.1 to 10000 mg/day and preferably from 1 to 100 mg/day as the dosage or intake of the active ingredient urolithin, and the daily dose or intake of these can be administered at once or by dividing into multiple times (e.g., 2 or 3 times).

### Dosage Form/Preparation Form

The dosage form of the sirtuin 6 activator of the present disclosure is not limited to a particular form. The dosage form of the sirtuin 6 activator in one embodiment of the present disclosure may be any of a solid form, a semi-solid form, or a liquid form, and can be appropriately set by those skilled in the art according to the type of active ingredient, the application and method of administration of the sirtuin 6 activator, and the like.

The sirtuin 6 activator in one embodiment of the present disclosure can be used, for example, as a component of a pharmaceutical, quasi-drug, cosmetic, food, or the like. For the additional component, those skilled in the art can appropriately select a component commonly used in pharmaceuticals, quasi-drugs, cosmetics, and foods. Specific examples of the additional component include pharmaceutically, sitologically, or cosmetically acceptable components, such as fillers, disintegrators, diluents, lubricants, fragrance imparting agents, colorants, sweeteners, flavoring agents, suspending agents, wetting agents, emulsifiers, dispersants, adjuvants, preservatives, buffers, binders, stabilizers, extenders, thickeners, pH adjusters, surfactants, coating agents, and nutrient components. One of these additional components may be selected and used, or two or more may be used in combination.

When the sirtuin 6 activator in one embodiment of the present disclosure is used as a component of a pharmaceutical, quasi-drug, cosmetic, or food, examples of the form of the pharmaceutical, quasi-drug, cosmetic, or food for oral administration include tablets, capsules, powders, granules, fine granules, sustained-release agents, solutions, syrups, jelly emulsions; and examples of the form for parenteral administration include injections, ointments, lotions, emulsions, creams, and powders. When the sirtuin 6 activator in one embodiment of the present disclosure is used as a component of a food, examples of the type of food include common foods and drinks, foods with health claims (including foods for specified health use, foods with nutrient function claims, and supplements), and foods for the sick.

Each aspect disclosed in the present specification can be combined with any other feature disclosed herein.

### EXAMPLES

Hereinafter, the present invention will be more specifically described by showing examples below, but each of configurations, their combinations, and the like in each embodiment are merely one example, and additions, omissions, substitutions, and other changes can be appropriately made without departing from the spirit of the present invention. The present disclosure is not limited by the embodiments and is limited only by the claims.

### Test Example 1: Preparation of Cells into which Promoter of Sirtuin 6 Gene is Introduced

The human SIRT6 promoter region (from −1105 to −1 bp; SEQ ID NO: 1) was amplified by PCR using KOD FX (TOYOBO, Tokyo, Japan) with genomic DNA derived from human fetal fibroblast TIG-1 as a template. Primers used were obtained by adding restriction endonuclease recognition sequences of AseI and NheI to the ends based on the reported base sequence information of the hSIRT6 promoter. Specifically, promoters designed as SIRT6p-AseI (ATTAATACTGCGCCCGGCTCACTCAC: SEQ ID NO: 2) and SIRT6p-NheI (GCTAGCCCTCGACTGCCCCACGGGAA: SEQ ID NO: 3) were used. KOD FX (TOYOBO) was used as the DNA polymerase, and PCR was performed under reaction conditions of 40 cycles with one cycle of 94° C. for 2 minutes, 98° C. for 10 seconds, and 68° C. for 1 minute.

The SIRT6 promoter fragment obtained by PCR was TA-cloned into a pGEM-T Easy Vector (Promega Corporation). In addition, the base sequence was confirmed by sequencing. The SIRT6 promoter fragment incorporated into the pGEM-T Easy vector was excised by digestion with restriction enzymes AseI and NheI and inserted into a site of EGFP-C3 (available from Takara Bio Inc.) from which the CMV promoter had been removed by AseI and NheI digestion, and pSIRT6p-EGFP was obtained.

Transfection was performed using HilyMax available from DOJINDO in accordance with its protocol. On the day before transfection, 6.0×10 5 cells of Caco-2 cells (human colon cancer-derived cells; obtained from RIKEN BioResource Research Center) were seeded in a 5-mL dish. On the day of transfection, pSIRT6p-EGFP (15 μg) was diluted with DMEM medium containing 10% FBS to a volume of 300 μL in a 1.5-mL tube, then 70 μL of Hilymax was added to the tube, and the mixture was incubated at room temperature for 20 minutes. Thereafter, the whole amount was added to Caco-2 cells and cultured for 48 hours.

Based on the fact that pSIRT6p-EGFP is resistant to the drug G418 (available from Wako Pure Chemical Industries, Ltd.), drug selection was performed by adding the drug G418 to transfected cells to a final concentration of 800 μg/mL. The culture medium was replaced with a fresh culture medium every 3 days, and G418 was added at the same concentration each time. Caco-2-SIRT6p-EGFP cells were obtained from this.

### Test Example 2: SIRT6-Enhancing Effect by Urolithin

Using Caco-2-SIRT6p-EGFP cells, a urolithin was examined for its enhancing effect on the promoter of the sirtuin 6 gene SIRT6. In addition, punicalin, puricalagin, and cafestol, which are known to have a sirtuin 1 gene-enhancing effect, were also examined for their enhancing effect on the promoter of the sirtuin 6 gene SIRT6.

Caco-2-SIRT6p-EGFP cells were seeded in a 96-well plate at 0.6×10 4 cells/well. The following day, 10 μM of the test component (urolithin A, punicalin, puricalagin, or cafestol) or a control (PBS) was added to each well. Two days after the addition, the culture solution was aspirated, then 100 μL of 4% paraformaldehyde was added to each well, and the well plate was allowed to stand at room temperature for minutes. After the well plate was let stand for 10 minutes, 4% paraformaldehyde was aspirated, and 100 μL of Hoechst 33342 solution (Dojindo) diluted 1/500 with PBS was added to each well, and the well plate was allowed to stand in a dark place at room temperature for 20 minutes. This was followed by aspiration and addition of 100 μL of PBS to each well, and the fluorescence intensity was measured with an IN Cell Analyzer 1000 (GE-Healthcare, Amersham Place, UK). The promoter activity was expressed as a relative ratio to the control activity.

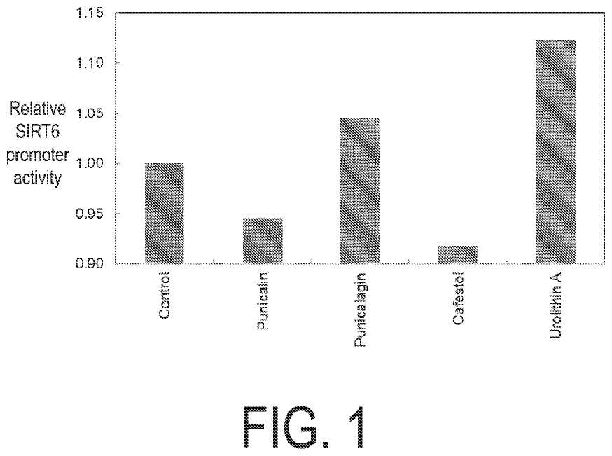

The obtained results (effect on the promoter activity of SIRT6 when the test component was added to Caco-2-SIRT6p-EGFP cells) are shown in FIG. 1. In FIG. 1, the vertical axis represents the relative SIRT6 promoter activity, and a higher value indicates a stronger promoter activity. As seen in FIG. 1, strong SIRT6 promoter-enhancing activity was confirmed for urolithin A, while punicalin, puricalagin, and cafestol, which are known to have a sirtuin 1 gene-enhancing effect, had no or little SIRT6 promoter-enhancing activity.

### Sequence Listing Free Text

SEQ ID NOs: 2 and 3 are primers.

1. A sirtuin 6 activator comprising a urolithin as an active ingredient.

2. The sirtuin 6 activator according to claim 1, wherein the urolithin is urolithin A.

3. The sirtuin 6 activator according to claim 1, for use as a therapeutic or prophylactic agent for hair loss.

4. The sirtuin 6 activator according to claim 1, for use as a hair follicle stem cell aging inhibitor.

5. The sirtuin 6 activator according to claim 1, for use as a therapeutic or prophylactic agent for gray hair.
